(19) United States
(12) Patent Application Publication
(10) Pub. No.: US 2015/0044091 A1
(43) Pub. Date: Feb. 12, 2015

(54) MICROWAVE STERILIZATION OF PHARMACEUTICAL CYANOACRYLATE ESTERS COMPOSITIONS

(71) Applicant: Sapheon, Inc., Morrisville, NC (US)

(72) Inventor: CARLOS R. MORALES, Morrisville, NC (US)

(21) Appl. No.: 14/453,493

(22) Filed: Aug. 6, 2014

Related U.S. Application Data
(60) Provisional application No. 61/862,901, filed on Aug. 6, 2013.

(51) Int. Cl. A61L 2/12

(52) U.S. Cl. CPC A61L 2/12
USPC 422/21; 422/186

(57) ABSTRACT

A method for sterilizing a cyanoacrylate composition comprises exposing it to microwaves. The method can further comprise post-heating the composition after microwave exposure, and/or cooling the composition after post-heating it. In addition, a system for sterilizing a cyanoacrylate composition comprises a sterilizing chamber, a microwave generator. The system can further comprise an infrared thermometer, a post-heating chamber, a heater thermometer, a cooling mechanism, and/or a belt. In some embodiments, the belt is configured to move sample vials containing cyanoacrylate to and from the sterilizing chamber, and to and from the post-heating chamber.

### CROSS-REFERENCE TO RELATED APPLICATIONS

This application claims the benefit under 35 U.S.C. §119(e) as a nonprovisional of U.S. Provisional Application No. 61/862,901, filed Aug. 6, 2013, titled MICROWAVE STERILIZATION OF PHARMACEUTICAL CYANOACRYLATE ESTERS COMPOSITIONS, the entirety of which is incorporated herein by reference.

### BACKGROUND

Cyanoacrylate esters compositions are well known for their fast and strong bonding properties. They have been used in different fields on a wide range of substrates. For example, they have been used as structural and industrial adhesives in consumer household products, in the automobile industry, and in many more industrial applications. In addition, cyanoacrylate compositions have been used in the medical and veterinary fields for wound management, tissue/organ repair, embolization and treatment of venous malformations, and venous reflux disease.

### SUMMARY

Disclosed herein are methods of sterilizing cyanoacrylate. The methods can include, in some embodiments, the steps of providing a first container comprising a first cyanoacrylate composition; moving the first container along a first pathway into a microwave chamber; exposing the first cyanoacrylate composition within the microwave chamber to microwave energy with a power of between about 0.1 kW to about 12 kW and at a temperature of less than about 130° C. for no more than about 30 seconds, such that a Bacillus subtilis count in the composition does not exceed 10⁻⁶; and moving the first container along a second pathway out of the microwave chamber. In some embodiments, moving the first container along the second pathway out of the microwave chamber comprises moving the first container into a conventional oven. The method can also include the steps of post-heating the composition in the oven for about 1 second to about 30 seconds; and substantially immediately after post-heating the composition, cooling the composition.

Also disclosed herein are systems for sterilizing a cyanoacrylate. In some embodiments, the system can comprise a sterilizing chamber; a microwave generator coupled to the sterilizing chamber; a heater maintenance post-heating chamber; a cooling mechanism; a belt configured to move the sample to and from the sterilizing chamber, and to and from the post-heating chamber; and a processing system in electronic communication with the microwave generator, the cooling mechanism, and the belt.

Disclosed herein are methods of sterilizing cyanoacrylate. The methods can include, in some embodiments, the steps of providing a first container comprising a first cyanoacrylate composition; moving the first container along a first pathway into a microwave chamber; exposing the first cyanoacrylate composition within the microwave chamber to microwave energy with a power of between about 0.1 kW to about 12 kW and at a temperature of less than about 190° C. for no more than about 30 seconds, such that a Bacillus subtilis count in the composition does not exceed 10⁻⁶; and moving the first container along a second pathway out of the microwave chamber.

In some embodiments, the methods can further include one or more of the following: moving the first container along the second pathway out of the microwave chamber comprises moving the first container into an oven; post-heating the composition in the oven for about 1 second to about 30 seconds; substantially immediately after post-heating the composition, cooling the composition; providing a second container comprising a second cyanoacrylate composition; moving the second container along the first pathway into the microwave chamber; exposing the second cyanoacrylate composition within the microwave chamber to microwave energy with a power of between about 0.1 kW to about 12 kW and at a temperature of less than about 190° C. for no more than about 30 seconds, such that a Bacillus subtilis count in the composition does not exceed 10⁻⁶; moving the second container along the second pathway out of the microwave chamber, where moving the second container along the first pathway into the microwave oven occurs after moving the first container along the second pathway out of the microwave chamber; the first cyanoacrylate composition is selected from the group consisting of: bis-2 cyanoacrylates; linear alkyl cyanoacrylates having from 2 to 12 carbons (ethyl, propyl, isopropyl, butyl, isobutyl, pentyl, hexyl, heptyl, octyl, nonyl, decyl, undecyl and dodecyl); branched 2-cyanoacrylates; 2-ethylhexyl, alcoxy cyanoacrylates; methoxyethyl, ethoxyethyl, butoxyethyl, methoxybutyl, ethoxymethyl, propoxyethyl, propoxymethyl, propoxypropyl, isopropoxyethyl, methoxypropyl, siloxyl cyanoacrylates; and butyl lactoyl, butyl glycoloyl, ethyllactoyl, ethylglycoloyl, and propiolactoyl cyanoacrylates; the first cyanoacrylate composition comprises an ionic inhibitor; the ionic inhibitor is selected from the group consisting of: sulfur dioxide, boron dioxide, chloro acetic acid, benzoic acid, hydrofluoric acid, dichloroacetic acid, trichloro acetic acid, acetic acid, nitric acid, sulfuric acid, tetrafluoroacetic acid, p-Toluenesulfonic acid, sultones, sulfonic acid, boron trifluoride, organic acids, alkyl sulfite, sulfolene, alkyl sulfoxide, and mercaptans; the first cyanoacrylate composition comprises a radical inhibitor; the radical inhibitor is selected from the group consisting of: butylated hydroxyanisol, butylated hydroxytoluene, t-butyl hydroxyquinone, pyrogallol, hydroquinone, hydroquinone monomethyl ether, benzoquinone and p-methoxyphenol; the first cyanoacrylate composition comprise thickeners and copolymers of the thickeners; the thickeners and the copolymers are selected from the group consisting of: polycyanoacrylates, polyglycolic acid, cellulose acetate fatty acid derivatives, cellulose acetate propionate, polypropiolactone, caprolactone copolymers, polyoxalates, fatty acid polymers, polyorthoesters, polyalkyl acrylates, polyalkyl methacrylates, polyoxypropylene polymers and block copolymers, polysugar and polysugar derivatives; the first cyanoacrylate composition comprises a radiopaque agent; the radiopaque agent is selected from the group consisting of: iodophenol derivatives, iodine complexes with pluronic polymers, gold and titanium particles, and mixtures thereof; the first cyanoacrylate composition comprises liquids, colloids or gels having a viscosity of between about 1 and about 10,000 centipoises; the cyanoacrylate composition comprises a dye; and/or the dye is selected from the group consisting of: anthracene, anthracene derivatives, FD&C violet No. 2, FD&C Red No. 3, FD&C Yellow No. 6, and FD&C Blue No. 2.

Disclosed herein are methods of sterilizing cyanoacrylate. The methods can include, in some embodiments, the step of exposing a cyanoacrylate composition to microwaves with a power of about 0.1 kW to about 12 kW, at temperature of about 50° C. to about 190° C., for a time period of no more than about 30 seconds, such that a Bacillus subtilis count in the composition does not exceed 10⁻⁶.

In some embodiments, the methods can further include one or more of the following: a frequency of the microwaves is about 2,450 MHz; the power ranges from about 0.5 kW to about 8 kW; the power ranges from about 0.1 kW to about 4 kW, about 0.5 kW to about 6 kW, or about 0.5 kW to about 12 kW; the microwave generator is configured to output a sterilization power ranging from about 0.5 kW to about 2 kW; the microwave generator is configured to output a sterilization power of about 0.6 kW; the microwave generator is configured to output a sterilization power of about 1.6 kW; the temperature is under 180° C.; the temperature is at about or under 184° C.; the temperature is at about or above 173° C.; the time period is no longer than about 9 seconds; the time period is for about 2 to about 9 seconds; the time period is no longer than about 5 seconds; the time period is no longer than about 2 seconds to about 3 seconds; substantially immediately after exposing the composition to microwaves, moving the composition to an oven; post-heating the composition in the oven; post-heating comprises post-heating the composition in the oven for no longer than about 30 seconds; post-heating comprises post-heating the composition in an oven for about 2 seconds to 4 seconds; post-heating comprises post-heating the composition in the oven for about 2 seconds to 3 seconds; substantially immediately after post-heating the composition, cooling the composition; cooling the composition comprises cooling the composition in a cooling chamber, in a water bath, in a chemical cooling bath, or with a fan; and/or substantially immediately after exposing the composition to the microwaves, exposing a second cyanoacrylate composition to microwaves with a power of about 0.1 kW to about 12 kW and a frequency of about 2,450 MHz, at temperature of about 50° C. to about 190° C., for a time period of no more than about 30 seconds, such that a Bacillus subtilis count in the second composition does not exceed 10⁻⁶.

Disclosed herein are systems for sterilizing cyanoacrylate. The systems can include, in some embodiments: a sterilizing chamber; a microwave generator coupled to the sterilizing chamber; a heater maintenance post-heating chamber; a cooling mechanism; a belt configured to move the sample to and from the sterilizing chamber, and to and from the post-heating chamber; and a processing system in electronic communication with the microwave generator, the cooling mechanism, and the belt.

In some embodiments, the systems can further include one or more of the following: an infrared thermometer configured to monitor a temperature of the sample in the sterilizing chamber; a heater thermometer configured to monitor a post-heat temperature of the sample in the post-heating chamber; the belt is configured to transfer sequential samples to the sterilizing chamber substantially immediately after transferring previous samples from the sterilizing chamber to the post-heating chamber; the microwave generator is configured to output a sterilization power ranging from about 0.1 kW to about 12kW; the microwave generator is configured to output a sterilization power ranging from about 0.1 kW to about 4 kW, about 0.5 kW to about 6 kW, or about 0.5 kW to about 12 kW; the microwave generator is configured to output a sterilization power ranging from about 0.5 kW to about 2 kW; the microwave generator is configured to output a sterilization power of about 0.6 kW; the microwave generator is configured to output a sterilization of about 1.6 kW; the microwave generator is configured to output a sterilization temperature ranging from about 50° C. to about 190° C.; the microwave generator is configured to output a sterilization temperature at about or under 184° C.; the microwave generator is configured to output a sterilization temperature at about or above 173° C.; the microwave generator is configured to output a sterilization temperature under 100° C.; the microwave generator is configured to output a sterilization condition for no longer than about 30 seconds; the microwave generator is configured to output a sterilization condition for no longer than about 9 seconds; the microwave generator is configured to output a sterilization condition for about 2 seconds to about 9 seconds; the microwave generator is configured to output a sterilization condition for no longer than about 5 seconds; the microwave generator is configured to output a sterilization condition for about 2 seconds to about 3 seconds; the post-heating chamber comprises an oven; the post-heating chamber is configured to post-heat the sample for no longer than about 30 seconds; the post-heating chamber is configured to post heat the sample for about 2 seconds to about 4 seconds; the post-heating chamber is configured to post heat the sample for about 2 seconds to about 3 seconds; the cooling mechanism is configured to cool the sample substantially immediately after the sample is post-heated in the post-heating chamber; the cooling mechanism is a cooling chamber, a fan, a water bath, or a chemical cooling bath; and/or an articulated arm to move samples to be sterilized within the system.

Disclosed herein are systems for sterilizing cyanoacrylate. The systems can include, in some embodiments: a sterilizing chamber; a microwave generator coupled to the sterilizing chamber; a belt configured to move the sample to and from the sterilizing chamber; and a processing system in electronic communication with the microwave generator and the belt.

In some embodiments, the systems can further include one or more of the following: an infrared thermometer configured to monitor a temperature of the sample in the sterilizing chamber; the microwave generator is configured to output a sterilization power ranging from about 0.1 kW to about 12 kW; the microwave generator is configured to output a sterilization power ranging from about 0.1 kW to about 4 kW, about 0.5 kW to about 6 kW, or about 0.5 kW to about 12 kW; the microwave generator is configured to output a sterilization power ranging from about 0.5 kW to about 2 kW; the microwave generator is configured to output a sterilization power of about 0.6 kW; the microwave generator is configured to output a sterilization of about 1.6 kW; the microwave generator is configured to output a sterilization temperature ranging from about 50° C. to about 190° C.; the microwave generator is configured to output a sterilization temperature at about or under 184° C.; the microwave generator is configured to output a sterilization temperature at about or above 173° C.; the microwave generator is configured to output a sterilization temperature under 100° C.; the microwave generator is configured to output a sterilization condition for no longer than about 30 seconds; the microwave generator is configured to output a sterilization condition for no longer than about 9 seconds; the microwave generator is configured to output a sterilization condition for about 2 seconds to about 9 seconds; the microwave generator is configured to output a sterilization condition for no longer than about 5 seconds; the microwave generator is configured to output a sterilization condition for about 2 seconds to about 3 seconds; a post-heating chamber; the belt configured to move the sample to and from the post-heating chamber; the belt is configured to transfer sequential samples to the sterilizing chamber substantially immediately after transferring previous samples from the sterilizing chamber to the post-heating chamber; a heater thermometer is configured to monitor a post-heat temperature of the sample in the post-heating chamber; the post-heating chamber comprises an oven; the post-heating chamber is configured to post-heat the sample for no longer than about 30 seconds; the post-heating chamber is configured to post heat the sample for about 2 seconds to about 4 seconds; the post-heating chamber is configured to post heat the sample for about 2 seconds to about 3 seconds; a cooling mechanism; the cooling mechanism is configured to cool the sample substantially immediately after the sample is post-heated in the post-heating chamber; the cooling mechanism is a cooling chamber, a fan, a water bath, or a chemical cooling bath; and/or an articulated arm to move samples to be sterilized within the system.

### BRIEF DESCRIPTION OF THE DRAWINGS

FIG. 1 illustrates an embodiment of a microwave sterilization system for a medical cyanoacrylate, according to some embodiments.

### DETAILED DESCRIPTION

Cyanoacrylate compositions require a number of additives to achieve specific applications. Currently, the methods of sterilizing cyanoacrylate compositions contribute to degradation of the additives and reduce the shelf life stability of cyanoacrylate compositions. To be used in the medical and veterinary fields, cyanoacrylate compositions can be sterilized so that the bacterial bioburden count or sterility assurance level of cyanoacrylate formulations does not exceed, for example, 10⁻⁶.

Generally, cyanoacrylate monomers have relatively low viscosity, which may cause the cyanoacrylate composition to spread into undesired areas. Thus, thickeners may be added to cyanoacrylate compositions to obtain formulations with the desired viscosity. However, the cyanoacrylate monomers and the compatible polymers which are used as thickeners are thermolabile. Therefore, when cyanoacrylate formulations with thickener additives are heated, the stability, shelf life, and effectives of the cyanoacrylate formulations could be compromised. Thus, a low temperature profile is generally preferable when sterilizing cyanoacrylate compositions by heat.

Cyanoacrylates, especially the lower homologues, are generally brittle. Thus, plasticizers are another group of additives that may be added to cyanoacrylate formulations. With the addition of plasticizers, cyanoacrylate formulations are more flexible and contour to body surfaces after polymerization. Examples of plasticizers include but are not limited to, alkyl esters of fatty acids such as myristates, triethylcitrate, alkyl laureates, alkyl stearates and alkyl succinates, citrate acetates, acetate acetyl citrates, phthalates, fatty acids acetates, benzoate esters, poly hydroxy branched aliphatic compounds, and phosphate esters. Other additives include radiopaque agents and dyes.

Anionic and radical inhibitors are another group of additives that may be added to cyanoacrylate formulations. For example, they may be added to prevent premature ionic polymerization of the cyanoacrylate compositions. Examples of acidic inhibitors include picric acid, sulfur dioxide, nitric oxide, hydrogen fluoride, acetic acid, sulfuric acid, nitric acid, lactic acid, ascorbic acid, and boron oxide phosphoric acid. In addition, radical inhibitors such as phenolic derivatives may be used. Some examples of phenolic derivatives include hydroquinone, BHA, BHT, catechol, and p-methoxyphenol.

Currently, methods of sterilizing cyanoacrylate compositions include irradiation by gamma rays and e-beam, visible light pulses having a wavelength of 390 nm to 780 nm, and dry heat sterilization. The gamma and e-beam procedures produce unstable formulations that require large doses of radical inhibitors. Furthermore, gamma and e-beam procedures can damage the plastic containers which contain the sealed compositions. Visible light irradiation is limited to the thickness of the cyanoacrylate composition and the thickness of the vials containing the formulation. In addition, production of UV radiation can induce radical polymerization of cyanoacrylate compositions. While dry heat sterilization produces stable compositions, dry heat sterilization affects the polymers and other additives used in some formulations. In addition, dry heat sterilization is often not reliably reproducible because dry heating is generally accomplished by ovens, which have hot spots and cold spots and thus heating may be inconsistent in different regions within the oven, thus leading to inconsistent heating.

Accordingly, some embodiments comprise systems and methods of sterilizing cyanoacrylate compositions by using microwave radiation. In some embodiments, microwave sterilization shortens the sterilization times dramatically, minimizing potential damaging effects that may be caused by long conventional dry heat cycles and ionizing sterilizers. The microwave sterilization system according to some embodiments operates with a variable power in the range of 0.1 kW to 12 kW or more and with a frequency of, for example, 2450 MHz. Microwave radiation comprises electromagnetic energy which falls at the lower end of the electromagnetic spectrum and has a measurement frequency of 300 MHz to 300 GHz with corresponding wavelengths of 1 cm to 1 m. The microwave region of the electromagnetic spectrum lies between the infrared and radio frequencies.

In some embodiments, cyanoacrylate compositions are sterilized by microwave radiation with a double killing effect. The primary killing effect can be generated in seconds by a dielectric heating effect with an instant production of heat that creates a primary lethal shock on bacteria and microbials. In some embodiments, the primary killing effect is followed by a post-heating exposure in a conventional oven or heating device to maintain a desired temperature for a short additional period of time. A second chemical killing effect can take place and can be produced by bactericidal and/or bacteriostatic properties of cyanoacrylate compositions, such as the interaction of liquid and gaseous cyanoacrylate compositions. Such effects can advantageously and synergistically kill dormant forms of microorganisms, including, for example, bacterial endospores. The short heating period time can limit the chemical degradation of cyanoacrylate monomers and also minimize the breakdown of the thermolabile polymer structure and other additives present in the composition.

Furthermore, the chemical, structural, and physical changes on the cyanoacrylate ester compositions are minimal according to some embodiments. Microwave sterilization can also have special applications on low to high viscous compositions. In addition, the embodiments disclosed herein may be capable of sterilizing cyanoacrylate compositions at lower temperatures compared to actual dry heat sterilization procedures. For example, some embodiments have short time exposure profiles, such as instant shock wave exposure profiles. Another advantage of some embodiments is based on the continuous process operation. For example, vials of cyanoacrylate can be sterilized one after another in some embodiments of the sterilization system. Furthermore, the embodiments disclosed herein allow for repeatable, consistent, and reproducible sterilization.

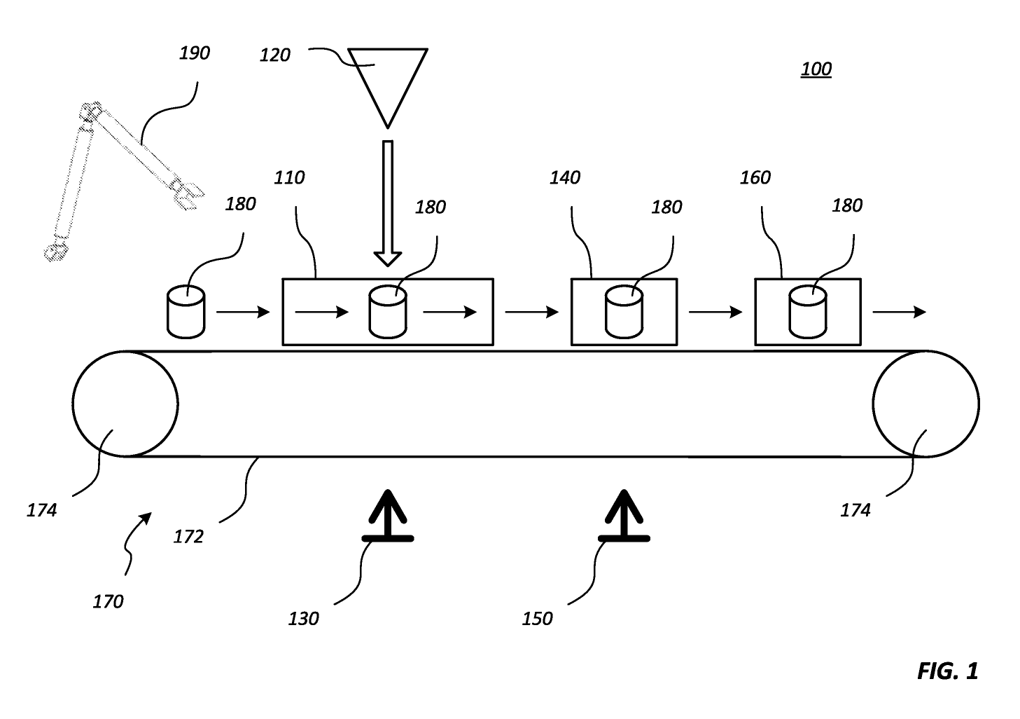

Referring to FIG. 1, some embodiments of a system 100 for sterilizing a cyanoacrylate composition include a sterilizing chamber 110, a microwave generator 120, an infrared thermometer 130, a post-heating chamber 140, a heater thermometer 150, a cooling mechanism 160, and a belt 170. In some embodiments, the sterilizing chamber 110 can comprise insulator materials such as polytetrafluoroethylene (PTFE) or ceramic. The system 100 may also include a processor, a voltage regulator, controls for the voltage regulator, an electric transformer, support cables, electric equipment, and/or other ancillary components for operating the system 100.

Also shown in FIG. 1 is a sample vial 180. In some embodiments, the sample vial contains a cyanoacrylate composition. The cyanoacrylate composition can comprise a monomer such as bis-2 cyanoacrylates, linear alkyl cyanoacrylates with 2 to 12 carbon (ethyl, propyl, isopropyl, butyl, isobutyl, pentyl, hexyl, heptyl, octyl, nonyl, decyl undecyl and dodecyl), branched 2-cyanoacrylates such as 2-ethylhexyl, alcoxy cyanoacrylates such as methoxyethyl, ethoxyethyl, butoxyethyl, methoxybutyl, ethoxymethyl, propoxyethyl, propoxymethyl, propoxypropyl, isopropoxyethyl, methoxypropyl, siloxyl cyanoacrylates, butyl lactoyl, butyl glycoloyl, ethyllactoyl, ethylglycoloyl, or propiolactoyl cyanoacrylates. In addition, in some embodiments the cyanoacrylate composition is a liquid, colloid, or gel and has a viscosity of about 1 centipoise to about 10,000 centipoises, such as between about 2 centipoises and about 4,000 centipoises.

Referring to FIG. 1, in some embodiments, the belt 170 comprises a continuous loop of material 172 that rotates about two or more pulleys 174 to form a conveyer system. For example, in some embodiments, the belt 170 is a conveyer belt that moves sample vials 180 in and out of a sterilizing chamber 110. In addition, the belt 170 can move sample vials to a post-heating chamber 140. Some embodiments also comprise an articulated arm 190. For example, the articulated arm 190 can move vials 180 to the sterilizing chamber 110 and/or place the vials onto the conveyer belt 170. The conveyor belt 170 can move the vial 180 from the sterilizing chamber 110 to the post heating chamber 140. The belt 170 and/or articulated arm 190 can be configured to move the sample 180 to and from different components of the system 100. In addition, the belt 170 and/or articulated arm 190 can be configured to move sequential samples 180 one after another, so that one sample 180 is sterilized one after another. In some embodiments, the belt 170 and the other components of the sterilizing system 100 operates continuously, so that the system 100 can sterilize a plurality of samples 180. Other automated assembly line-type mechanisms for sequentially sterilization of a plurality of samples can also be used.

In use, the belt 170 and/or articulated arm 190 can move a sample 180 to the sterilizing chamber 110 where it is exposed to microwaves by the microwave generator 120. Some embodiments include an infrared or other thermometer 130 for monitoring the temperature of the sample 180 during microwave irradiation. After the sample 180 is exposed to microwaves, the belt 170 and/or articulated arm 190 can move the sample to a post-heating chamber 140. In some embodiments, the post-heating chamber 140 is an oven or another heating device. A heater thermometer 150 can be included in order to monitor the temperature of the sample 180 during post-heating. Next, the belt 170 or articulated arm 190 can move the sample 180 to a cooling mechanism 160. In some embodiments, the cooling mechanism 160 is a fan or a cooling bath. Because the system 100 is capable of continuous operation, after the belt 170 and/or articulated arm 190 moves a sample 180 from the sterilizing chamber 110 to the post-heating chamber 140, a second sample can be moved to the sterilizing chamber 110. After the first sample 180 is moved from the post-heating chamber 140 to the cooling mechanism 160, the second sample can be moved from the sterilizing chamber 110 to the post-heating chamber 140. In this manner, the system 100 can continuously and sequentially sterilize a multitude of samples 180.

The sample vial 180 can be moved to and from the components of the system 100 in other ways as well besides via a conveyer belt 170. For example, in some embodiments, the sterilizing chamber 110 is configured vertically, and the system 100 includes vertical feeding arms. Further, the sterilizing chamber 110 can be configured to rotate in some embodiments. The post-heating chamber 140 can similarly be configured to rotate, and another mechanism provided to deliver the sample vial to a cooling system (e.g., cooling mechanism 160).

Referring to FIG. 1, the microwave generator 120 can be operably connected to the sterilizing chamber 110 so that sample vials 180 in the sterilizing chamber 110 are exposed to focused microwaves generated by the microwave generator 120. In some embodiments, the microwave generator 120 can generate sterilizing conditions in the chamber 110. For example, in some embodiments the microwave generator 120 can output a power wattage between 0.1 kW to 12 kW with a frequency of 2450 MHz, at a temperature between about 50° C. to 200° C., for 30 seconds or less. Under these conditions, the Bacillus subtilis count in cyanoacrylate compositions SAL does not exceed 10⁻⁶ according to some embodiments. Other microwave frequencies besides 2450 MHz are possible, such as, for example, about 915 MHz, about 5800 MHz, about 300 MHz to about 20 GHz, or about 915 MHz to about 2450 MHz. Alternatively, a signal having a frequency ranging from about 2.45 GHz to about 10 GHz may also be utilized.

In some embodiments, the sample vial 180 can be sterilized at 2 kW to 8 kW, 0.5 kW to 4 kW, 0.5 kW to 6 kW, or 0.5 kW to 12 kW. In some embodiments, vials 180 can be sterilized at a power of 0.5 kW to about 2 kW, such as about 1 kW, 1.2 kW, 1.4 kW, 1.6 kw, or 1.8 kW. Furthermore, the power can be variable and adjustable, and/or pulsed in some embodiments. In addition, the sample vial 180 can be sterilized at low temperature profiles, such as a temperature below or about 100° C., in order to prevent degradation of the certain cyanoacrylate composition containing thermolabile additives. A temperature below 100° C. in some cases also prevents premature polymerization of the cyanoacrylate composition. In some embodiments, the sample vial 180 can be sterilized at higher temperature profiles such as above or about 170, 171, 172, or 173° C. Unlike conventional ovens, focus microwaves, such as those with a frequency of 2450 MHz, do not create hot or cold spots. Thus, microwave irradiation can be uniformly applied throughout the sample 180 and the results are consistent, repeatable, and reproducible. When the sample vial 180 is exposed to microwaves, an infrared thermometer 130 can be utilized to monitor the temperature of the sample vial 180.

In some embodiments, the cyanoacrylate composition 180 is exposed to microwaves for a short period of time, such as about 30 sec, 25 sec, 20 sec, 15 sec, 10 sec, 9 sec, 8 sec, 7 sec, 6 sec, 5 sec, 4 sec, 3 sec, 2 sec, or 1 second or less, or ranges encompassing any two of the aforementioned time values. The sterilization duration in the sterilization chamber 110 can also be 1 to 4 seconds in some embodiments, and 2 to 6 seconds in other embodiments. In some embodiments, the sterilization duration is based on the type of cyanoacrylate composition 180, the thickness of the sample vial container 180, and/or the volume of the cyanoacrylate composition 180. For example, a thicker sample vial container and an increased volume of cyanoacrylate could potentially result in increased sterilization duration. After cessation of the microwave beam, energy still causes dipole heating. Thus, it may be desirable to shorten the sterilization duration time as much as possible. In some embodiments, the sample vial 180 may require additional sterilization, such as in a conventional dry heat oven or heating device.

In some embodiments, the sample vial 180 can be transferred to a post-heating chamber 140, such as an oven, substantially immediately after the sample vial 180 is exposed to microwave beams in the sterilization chamber 110. In some embodiments, the post-heat temperature is less than about 200° C., 190° C., 180° C., 170° C., 160° C., 150° C., 140° C., 130° C., 120° C., 110° C., 100° C., 90° C., or less. The sample vial 180 is post-heated for no longer than 30 seconds, and preferably for 2-3 seconds, according to some embodiments. During this time, a heater thermometer 150 can be utilized to monitor the temperature of the sample vial 110.

In some embodiments, microwave sterilization using predetermined parameters such as those disclosed herein can fairly precisely control the final viscosity of a formulation, such as within about 10%, or about 5% of the pre-sterilization viscosity. In some embodiments, an overly high viscosity at room temperatures (e.g., at least about 2100 cP, 2200 cP, 2300 cP, or more) can lead to progressive polymerization of the adhesive leading to an unusable specimen. If these parameters are not controlled, the viscosity of the composition following heating, mixing, and/or cooling can vary unpredictably, potentially leading to yield and quality control issues, among others. In some embodiments, the viscosity of the composition following heating can be controlled such that it is between about 2 to 1000 cP, between about 1,000-2,000 cP, between about 1,100 cP-2,000 cP, or between about 1,400-1,800 cP.

Some embodiments further comprise a cooling mechanism 160, for cooling the sample vial 180 substantially immediately after it is post-heated in the post-heating chamber 140. The cooling mechanism 160 can be a fan, a water bath, a chemical cooling bath, a cooling chamber, or any other suitable mechanism.

An example embodiment of sterilizing cyanoacrylate includes sterilizing a thick cyanoacrylate adhesive composition including Cellulose Acetate Butyrate (CAB) with, for example, a melting point of about 135° C. and a glass transition temperature of about 115° C. In some embodiments, the melting point can range from about 100 to 170° C. The glass transition temperature can range from about 80 to 150° C. The thick cyanoacrylate composition with a CAB polymer can have a viscosity of about 1655 cP at 25° C. (e.g., about room temperature). The thick cyanoacrylate composition with a CAB polymer can have other viscosities as discussed herein. A pure cyanoacrylate monomer and/or thin cyanoacrylates (e.g., viscosity of about 3 cP at 25° C.) may also be sterilized according to embodiments disclosed herein.

In some embodiments, a cyanoacrylate sample (e.g., a thick cyanoacrylate adhesive composition including CAB with a melting point of about 135° C., a glass transition temperature of about 115° C., and a viscosity of 1655 cP at 25° C.) is sterilized with exposure to microwaves for at least about 2 seconds at a power of about 1.6 kW to attain a sterilizing temperature of at least about 173° C. without exposure to post-heating. For example, sterilization can be achieved with the foregoing or other operating parameters for a cyanoacrylate sample having biological indicators with a bioburden of 10⁻⁶ Bacillus subtillis spores at a microwave exposure that generates a sterilization temperature of at least about 173° C.

The microwave exposure time for sterilization may vary from about 1, 1.2, 1.4, 1.6, 1.8, 2 or less to 9 or more seconds depending on, for example, microwave power output. For example, sterilization of cyanoacrylate can be achieved with a microwave power of about 0.6 kW and an exposure time of about 9 seconds to attain a temperature of about 176° C. In some embodiments, the cyanoacrylate sample that has been exposed to a microwave power of about 0.6 kW for about 9 seconds can be further exposed to post heating for about 4 seconds to attain a sterilization temperature of about 184° C. to, for example, further sterilize the cyanoacrylate.

Accordingly, microwave sterilization temperature can be controlled by controlling the microwave power and sample exposure time to microwave radiation. The microwave power and exposure time can be inversely related to each other. The lower the relative microwave power, the longer the exposure time should be to attain sterilization of cyanoacrylate. The higher the relative microwave power, the shorter the exposure time can be to attain sterilization of cyanoacrylate.

In some embodiments, by controlling and achieving a desired or predetermined sterilization temperature, cyanoacrylate may be satisfactorily or sufficiently sterilized without exposure to post-heating (e.g., post-heating chamber 140).

With or without exposure to post-heating, sterilization of cyanoacrylate according to embodiments disclosed herein can minimally/insignificantly affect or substantially not affect viscosity of the cyanoacrylate sample. For example, the thick cyanoacrylate adhesive composition including CAB with a melting point of about 135° C., a glass transition temperature of about 115° C., and a viscosity of 1655 cP at 25° C. that is sterilized as discussed herein may have a sterilized viscosity of about 1550 cP to 1655 cP, including about 1600 cP to 1650 cP.

In some embodiments, cyanoacrylate is contained in sealed sample vial 180 made of glass that is compatible with cyanoacrylate. For example, the sample vial 180 can be made of type 1 borosilicate glass treated or coated with silicone, Lewis acids moieties, sulfur dioxide, boron oxide, sulfuric acid, nitric acid, acetic acid, or an anionic and radical inhibitor moiety. In other embodiments, the cyanoacrylate composition can be sealed in a sample vial 180 made of plastic, such as polyethylene, polypropyelene, polytetrafluoroethylene, a cyanoacrylate resistant fluorinated plastic, or a cyanoacrylate resistant unfluorinated plastic. In other embodiments, the cyanoacrylate composition can be sealed in a sample vial 180 made of metal such as aluminum, tin, or any other suitable metal alloy. In addition, the samples can be sealed in the described plastic or metal containers and placed in a second empty container made of plastic, metal, or glass and sterilized by microwave sterilization.

The embodiments disclosed herein can sterilize cyanoacrylate compositions without degrading additives such as ionic inhibitors, radical inhibitors, thickeners, radiopaque agents, and dyes. In addition, cyanoacrylate compositions can be sterilized with minimal chemical, structural and physical changes on the cyanoacrylate ester compositions.

Examples of ionic inhibitors include sulfur dioxide, boron dioxide, chloro acetic acid, benzoic acid, hydrofluoric acid, dichloroacetic acid, trichloro acetic acid, acetic acid, nitric acid, sulfuric acid, tetrafluoroacetic acid, p-Toluenesulfonic acid, sultones, sulfonic acid, boron trifluoride, organic acids, alkyl sulfite, sulfolene, alkyl sulfoxide, and mercaptans.

Examples of radical inhibitors and their mixtures include butylated hydroxyanisol, butylated hydroxytoluene, t-butyl hydroxyquinone, pyrogallol, hydroquinone, hydroquinone monomethyl ether, benzoquinone, and p-methoxyphenol.

Examples of thickeners include polycyanoacrylates, polyglycolic acid, cellulose acetate fatty acid derivatives, cellulose acetate propionate, cellulose acetate butyrate, polypropiolactone, caprolactone copolymers, polyoxalates, fatty acid polymers, polyorthoesters, polyalkyl acrylates, polyalkyl methacrylates, polyoxypropylene polymers and block copolymers, polysugar, and polysugar derivatives.

Examples of radiopaque agents include iodophenol derivatives, iodine complexes with pluronic polymers (polyoxypropylene), gold and titanium particles, and other metals and their mixtures.

Examples of dyes include derivatives of anthracene, D&C violet No. 2, FD&C Red No. 3, FD&C Yellow No. 6, and FD&C Blue No. 2.

The embodiments disclosed herein describe cyanoacrylate compositions merely as an example. One of skill in the art will appreciate that embodiments may be used with a wide variety of pharmaceuticals as well as other compounds, including but not limited to polymerized compounds such as PMMA.

After completing the sterilization process—for example after exposing the sample vial 180 to microwave radiation, after post-heating the sample, and after cooling the sample—the process can be verified by utilizing vials with wires and cotton strand biological indicators embedded with a 10⁻⁶ Bacillus subtilis bacterial population spores inside sealed vials with the cyanoacrylate compositions. In addition, the sterilization process can be assayed by transferring the sample vial 180 to a sterile aldose solution and a sterile tryptic soy broth, incubating the composition for a predetermined amount of time at about 37° C., and monitoring bacterial growth.

The terms “approximately”, “about”, and “substantially” as used herein represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

Although certain embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or subcombinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above.

What is claimed is:

1. A method of sterilizing cyanoacrylate, the method comprising:
providing a first container comprising a first cyanoacrylate composition;
moving the first container along a first pathway into a microwave chamber;
exposing the first cyanoacrylate composition within the microwave chamber to microwave energy with a power of between about 0.1 kW to about 12 kW and at a temperature of less than about 190° C. for no more than about 30 seconds, such that a Bacillus subtilis count in the composition does not exceed 10⁻⁶; and
moving the first container along a second pathway out of the microwave chamber.

2. The method of claim 1, wherein moving the first container along the second pathway out of the microwave chamber comprises moving the first container into an oven, and
wherein the method further comprises:
post-heating the composition in the oven for about 1 second to about 30 seconds; and
substantially immediately after post-heating the composition, cooling the composition.

3. The method of claim 1, further comprising:
providing a second container comprising a second cyanoacrylate composition;
moving the second container along the first pathway into the microwave chamber;
exposing the second cyanoacrylate composition within the microwave chamber to microwave energy with a power of between about 0.1 kW to about 12 kW and at a temperature of less than about 190° C. for no more than about 30 seconds, such that a Bacillus subtilis count in the composition does not exceed 10⁻⁶; and
moving the second container along the second pathway out of the microwave chamber, wherein moving the second container along the first pathway into the microwave oven occurs after moving the first container along the second pathway out of the microwave chamber.

4. A method of sterilizing cyanoacrylate, the method comprising:
exposing a cyanoacrylate composition to microwaves with a power of about 0.1 kW to about 12 kW, at temperature of about 50° C. to about 190° C., for a time period of no more than about 30 seconds, such that a Bacillus subtilis count in the composition does not exceed 10⁻⁶.

5. The method of claim 4, wherein the microwave generator is configured to output a sterilization power ranging from about 0.5 kW to about 2 kW.

6. The method of claim 5, wherein the microwave generator is configured to output a sterilization power of about 0.6 kW.

7. The method of claim 5, wherein the microwave generator is configured to output a sterilization power of about 1.6 kW.

8. The method of claim 4, wherein the temperature is at about or above 173° C.

9. The method of claim 4, wherein the time period is no longer than about 9 seconds.

10. The method of claim 4, further comprising:
substantially immediately after exposing the composition to microwaves, moving the composition to an oven; and
post-heating the composition in the oven.

11. The method of claim 10, wherein post-heating comprises post-heating the composition in the oven for no longer than about 30 seconds.

12. The method of claim 11, wherein post-heating comprises post-heating the composition in the oven for about 2 seconds to 3 seconds.

13. The method of claim 10, further comprising:
substantially immediately after post-heating the composition, cooling the composition.

14. The method of claim 13, wherein cooling the composition comprises cooling the composition in a cooling chamber, in a water bath, in a chemical cooling bath, or with a fan.

15. A system for sterilizing a cyanoacrylate, the system comprising:
a sterilizing chamber;
a microwave generator coupled to the sterilizing chamber;
a belt configured to move the sample to and from the sterilizing chamber; and
a processing system in electronic communication with the microwave generator and the belt.

16. The system of claim 15, further comprising an infrared thermometer configured to monitor a temperature of the sample in the sterilizing chamber.

17. The system of claim 15, further comprising a post-heating chamber.

18. The system of claim 17, wherein the post-heating chamber comprises an oven.

19. The system of claim 15, further comprising a cooling mechanism.

20. The system of claim 19, wherein the cooling mechanism is a cooling chamber, a fan, a water bath, or a chemical cooling bath.
